(19) United States
(12) Patent Application Publication
(10) Pub. No.: US 2009/0204030 A1
(43) Pub. Date: Aug. 13, 2009

(54) METHOD AND SYSTEM FOR MONITORING THE FUNCTIONAL USE OF LIMBS

(75) Inventors: Andreas Brauers, Aachen (DE); Gerd Lanfermann, Aachen (DE); Olaf Such, Aachen (DE); Richard Daniel Willmann, Siegburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/282,016

(22) PCT Filed: Mar. 8, 2007
(86) PCT No.: PCT/IB07/50775

§ 371 Date: Sep. 8, 2008

(30) Foreign Application Priority Data
Mar. 8, 2006 (EP) 06110868.4

(51) Int. Cl. A61B 5/11

(52) U.S. Cl. 600/595

(57) ABSTRACT

The invention relates to the area of rehabilitation for patients with motor disabilities, especially for hemiplegic patients. With the present invention a long term, e.g. day and night, patient activity monitoring can be established in a home environment. A core idea of the invention is to evaluate the functional use of a limb by determining the synchronicity of the patient's movements depending on the movement's cyclicity. With this approach a reliable assessment of limb usage can be made based on the daily living activities of the patient. With the present invention the progress of rehabilitation can be monitored and guidance can be provided to therapists and patients about the state of the rehabilitation process. It further makes them aware of a lack of limb usage.

The present invention relates to a method and system for monitoring the functional use of limbs. Furthermore the invention relates to a computer program.

Half-sided paralysis (hemiplegy) is one of the most common symptoms of neurological disorders, e.g. stroke. Rehabilitation must be aimed at restoring the functional use of the limb, i.e. in activities of daily life. The use of disabled limb is the best approach to achieve rehabilitation success. However, many patients prefer not to use the disabled limb.

It is well known that people with motor disabilities tend to use the impaired part of the body to a lesser extent and to overuse other parts of the body. For example, a healthy leg is usually loaded more than a non-healthy leg during walking. This imbalance helps the impaired person to compensate for the disability to some extent. In the long term this may lead to injuries in the healthy limb due to overuse and further weakening of the impaired limb or part of the body.

Usually, rehabilitation exercises are made to strengthen neglected limbs and establish the most natural motion pattern possible to be performed by the individual patient. To some extent the patient using mirrors or videotaping can continue these exercises at home. There are a number of disadvantages with such systems: The time of monitoring of the movements is limited to the time in which these rehabilitation exercises are performed, while daily use of limbs is not monitored. The patient's movements are only analyzed when he carries out artificial therapeutic movements. Progress in these movements is known not to translate to functional movements in everyday life. A therapist/patient relationship requires the visit of an institution (rehabilitation centre, clinic). When the patient observes his own posture and asymmetric movement, he may oversee certain defects. Slowly developing defects are not noticed (by both the professional and the patient) since they are regarded as a typical performance. It is difficult for a human to judge gradual changes in the physical performance when no historic data areas available for reference.

Technical solutions addressing asymmetries in motion patterns are mostly based on video monitoring, which restricts monitoring to dedicated places and to performing special tasks. Another way is to use shoes with soles that sense the symmetry of weight loads. This method is however restricted to assessing under/overuse of legs and cannot be used to analyze motion patterns, e.g. during walking.

It is an object of the present invention to provide a technique, which allows easy monitoring of the correct functional use of a patient's limb.

This object is achieved according to the invention by a method of monitoring the functional use of limbs, comprising the steps of:
performing a permanent and unsupervised measuring of movement data, using a measuring unit, with at least parts of said measuring unit being carried by the monitored patient,
analyzing said movement data,
determining the movement's cyclicity C,
determining the movement's synchronicity S depending on the movement's cyclicity C, and
storing the movement's synchronicity value S and/or notifying the patient about the findings.

The object of the present invention is also achieved by a system for monitoring the functional use of limbs, comprising a measuring unit, with at least parts of said measuring unit being carried by the monitored patient, said measuring unit being adapted to perform a permanent and unsupervised measuring of movement data, and a data unit, said data unit being adapted
to analyze said movement data,
to determine the movement's cyclicity C,
to determine the movement's synchronicity S depending on the movement's cyclicity C, and
to store the movement's synchronicity value S and/or notify the patient about the findings.

All system means are adapted to carry out the method according to the present invention. All devices and units, e.g. the measuring unit and the data unit, are constructed and programmed in a way that the procedures for obtaining data, for data processing and system control run in accordance with the method of the invention.

The object of the present invention is also achieved by a computer program to be executed in a computer, said computer program being adapted for monitoring the functional use of limbs, using movement data obtained during a permanent and unsupervised measuring performed by a measuring unit, with at least parts of said measuring unit being carried by the monitored patient, the program comprising:
computer instructions to analyze said movement data,
computer instructions to determine the movement's cyclicity C,
computer instructions to determine the movement's synchronicity S depending on the movement's cyclicity C, and
computer instructions to store the movement's synchronicity value S and/or to notify the patient about the findings,

when the computer program is executed in the computer.

The technical effects necessary according to the invention can thus be realized on the basis of the instructions of the computer program in accordance with the invention. Such a computer program can be stored on a carrier such as a CD-ROM or it can be available over the Internet or another computer network. Prior to execution, the computer program is loaded into the computer by reading the computer program from the carrier, for example by means of a CD-ROM player, or from the Internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e.g. using one or more integrated circuits.

The invention relates to the area of rehabilitation for patients with motor disabilities, especially for hemiplegic patients. With the present invention a long-term, e.g. day and night, patient activity monitoring can be established in a home environment. A core idea of the invention is to evaluate the functional use of a limb by determining the synchronicity of the patient's movements depending on the movement's cyclicity. With this approach a reliable assessment of limb usage can be made based on the daily living activities of the patient. With the present invention the progress of rehabilitation can be monitored and information can be provided to therapists and patients about the state of the rehabilitation process. It further makes them aware of a lack of limb usage. It is also possible to give feedback to the patient (or the therapist) in real-time, i.e. while certain movements are being carried out.

These and other aspects of the invention will be further elaborated on the basis of the following embodiments which are defined in the dependent claims.

In contrast to prior art technology, in which the evaluation is based upon the overall amount of motion of the affected limbs, the present invention provides an improved way of evaluating movement information. According to a preferred embodiment of the invention the step of analyzing the movement data includes classification of the patient's movement. Such classifying includes the determination of movement patterns, in particular different types of cyclic movements, e.g. walking, running. It may be assumed that cyclic movements are always functional. By restricting the evaluation to cyclic movements, the evaluation result is more valuable for assessing the progress of rehabilitation, compared to former known evaluation techniques.

Preferably the cyclicity threshold value C₀ is set depending on such a movement classification. In other words, the threshold is chosen depending on the kind of limb movement, e.g. walking or running. Hence, different limb movements can be addressed differently. This enables a sophisticated differentiation and evaluation process.

In a preferred embodiment of the invention the movement's synchronicity S is determined only if the movement's cyclicity value C exceeds the cyclicity threshold value C₀. In other words, the movement's synchronicity is determined only if the patient's movement is cyclic. Only in this case is a symmetric movement expected. This approach of data filtering leads to a simplification of monitoring both with regard to the monitoring method and with regard to the monitoring system and thus to a significant reduction of monitoring costs. Furthermore, since the symmetry of limb movements is evaluated with respect to certain cyclic movements only, the evaluation is not distorted by non-functional movements.

In order to quantify the movement's synchronicity, a synchronicity threshold value S₀ is preferably set depending on the movement classification, thereby taking into account that different synchronicity values hold for different movements. In a further embodiment of the invention the synchronicity threshold value S₀ is—at least from a particular monitoring time on—dynamically adapted depending on prior and/or current synchronicity values S. The therapeutic progress of a specific patient can thus be taken into account.

In another preferred embodiment of the invention the movement's synchronicity S is compared with the synchronicity threshold value S₀ and the patient is notified only in case the movement's synchronicity value S falls below the synchronicity threshold value S₀. If the movement's synchronicity value S remains above the synchronicity threshold value S₀, there is no need for notification, since the target synchronicity, i.e. a limb movement according to the required rehabilitation process, is reached.

In another preferred embodiment of the invention a biofeedback approach is implemented, in which the kind of notification depends on the determined synchronicity S. For example audio and/or video signaling or mechanical/electrical stimulation of the corresponding limb can be used. Furthermore, the feedback is quantified in a sense that the feedback is differentiated depending on the determined synchronicity S with simultaneous consideration of the determined kind of patient movement. Such a feedback leads to a better acceptance of the monitoring method by the patient.

In yet another embodiment of the invention the time of notification depends on the actual movements of the patient. In other words, the patient is not notified about progresses or regresses of a certain movement, e.g. about the activity status of his left arm during running, when he actually has just stopped running and now drinks a cup of tea. However, as soon the patient starts running again, he might be notified accordingly.

In a preferred embodiment of the invention the measuring unit comprises a number of inertial sensors in order to obtain “dynamic” movement data. This is in contrast to prior art technique, where “static” data, like body positions etc., are used. Different types of inertial sensors might be used. However, the use of accelerometer, gyroscopes and magnetometer devices is preferred. In order to monitor the symmetry of the movement patterns of the patient, these sensor devices will preferably be worn by the patient on the affected limb and its counterpart, e.g. left arm (affected) and right arm (healthy).

The present invention relates in particular to a system and method for monitoring asymmetries in cyclic movements of a patient's limbs, comprising the use of accelerometers attached to the limbs and trunk and analyzing the movements for symmetry. The analysis is carried out by means of a data processing device, which executes algorithms to evaluate integral symmetry of limb movement or real-time characterization of motion patterns in terms of symmetry. The system comprises feedback facilities to inform the patient about the success or failure to use limbs symmetrically. Further, the system may comprise feedback facilities for a physiotherapist to control patient compliance and success of therapy. With the invention it is possible to establish long-term, unsupervised movement monitoring with a patient-worn system, which allows easy surveillance of the correct functional use of a patient's limb.

These and other aspects of the invention will be described in detail hereinafter, by way of example, with reference to the following embodiments and the accompanying drawings; in which:

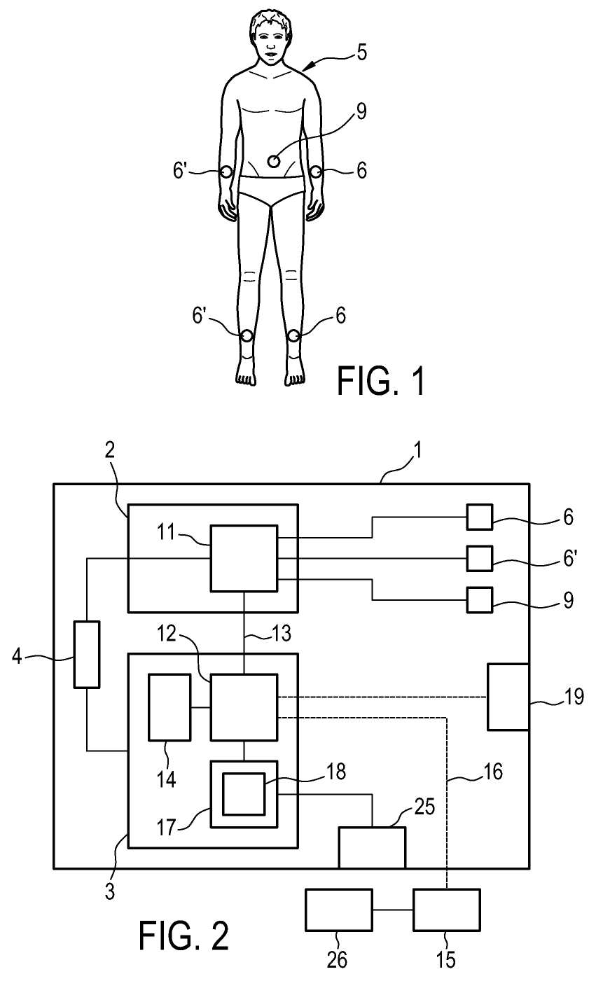

FIG. 1 shows a schematic illustration of a user wearing the system according to the present invention,

FIG. 2 shows a schematic illustration of the system components according to the present invention,

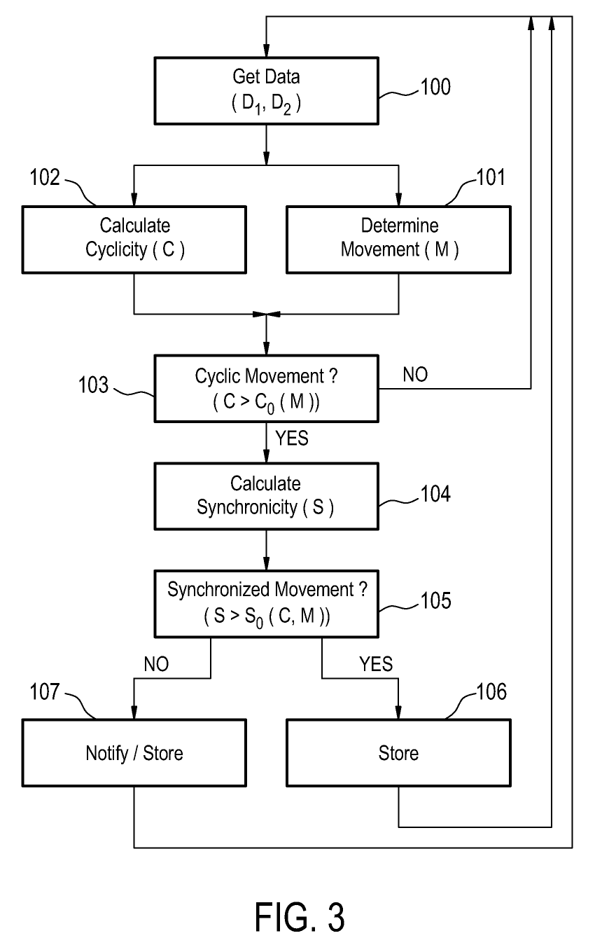

FIG. 3 shows a simplified flowchart of the method according to the invention, and

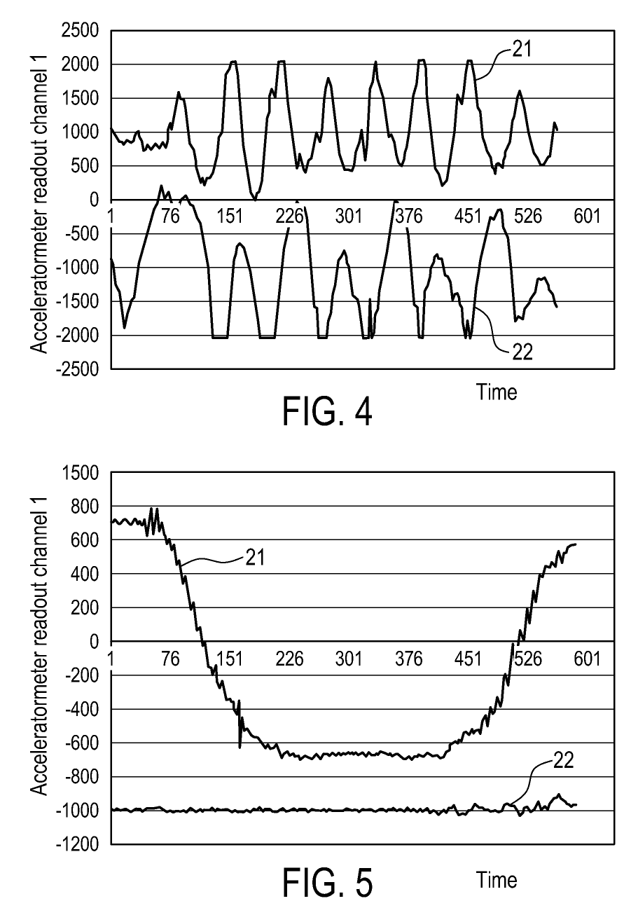

FIG. 4 shows a sensor readout while the patient is drinking tea,

FIG. 5 shows a sensor readout while the patient walks,

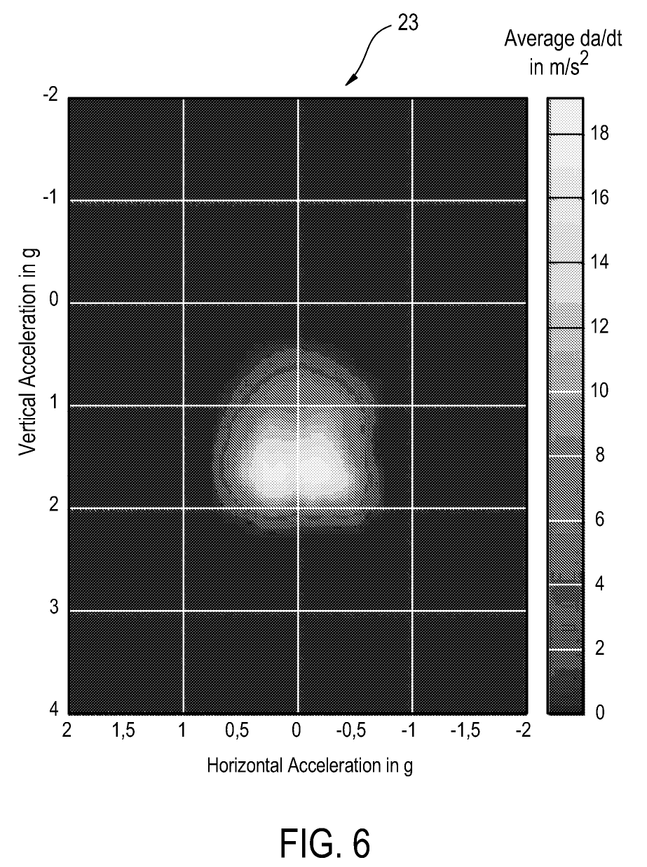

FIG. 6 shows a first accelerometer histogram,

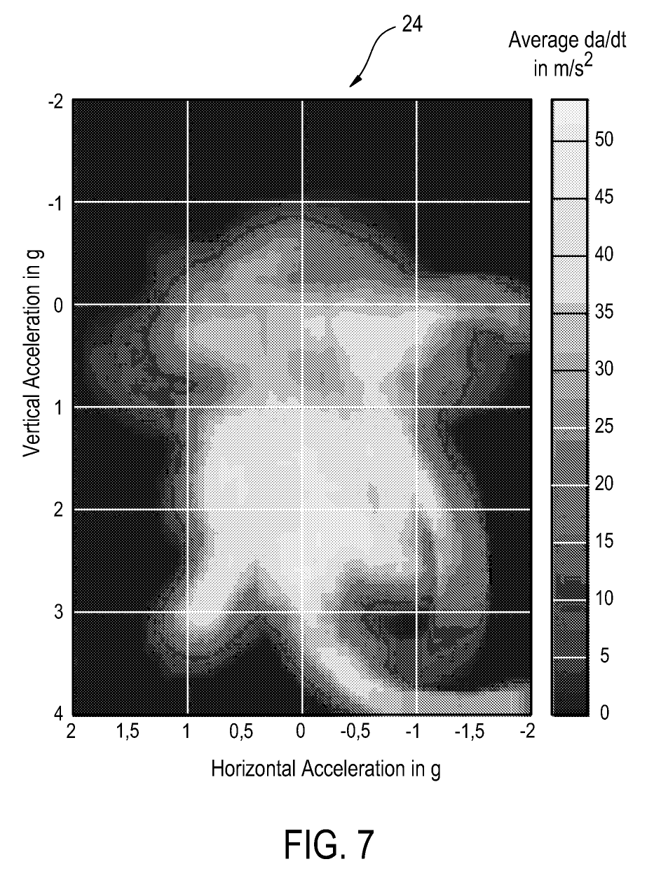

FIG. 7 shows a second accelerometer histogram.

A monitoring system according to the present invention is illustrated in FIGS. 1 and 2. The system comprises a measuring unit 2 and a data unit 3. At least one power source, e.g. a battery 4, is part of the system 1.

The measuring unit 2 is adapted to perform a permanent and unsupervised measuring of a patient's 5 movement data. The measuring unit 2 comprises a number of measuring sensors to measure the movement of the patient 5. As measurement sensors two or more inertial sensors in the form of accelerometers 6 are used. For the purpose of the present invention two accelerometers 6, 6′ are positioned at least on the affected limb 7 and the corresponding healthy limb 8. The accelerometers 6, 6′ are adapted to provide signals within a measuring range of +/−2 g with a resolution of some 10⁻³ g at a sample rate of 204.8 MHz. However, other types of movement sensors may be employed. The accelerometers 6, 6′ are worn at symmetric positions on two equal body parts (e.g. arms, hands, legs, hips). A third accelerometer 9 is worn at a neutral position along the z-axis of the patient's 5 body to serve as a reference. Preferably, the additional third accelerometer 9 is attached to the torso of the patient 5.

The accelerometers 6, 6′, 9 are preferably worn as bracelets around the arm or leg or integrated into the patient's underwear etc. However, they can as well be worn as patches or implants. The frequency at which sensor data are measured, may be fixed or adapted to the movement. E.g. a fast walk might need a higher sampling rate than during a slow movement.

Further, the measuring unit 2 comprises data communication equipment 11 for transmitting measuring data to the data unit 3. Each accelerometer 6, 6′, 9 is adapted to transmit its movement profile. The movement profile consists of sensor acceleration data in x-, y- and z-direction and preferably a time stamp.

The data unit 3 comprises data communication equipment 12 adapted to receive data from the measuring unit 2 and preferably further adapted to send data to further external devices. In other words, the data unit 3 is adapted to collect measurement data from the three or more accelerometers 6, 6′, 9. Data are continuously streaming from the sensors to the data unit 3. The data are transmitted to the data unit 3 either by cable 13 or by using wireless communication line. The data unit 3 either stores the data for off-line processing or immediately analyses the data as described below in more detail. For data storage the data unit 3 comprises an integrated storage device 14. Alternatively, data may be transmitted from the measuring unit 2 and/or the data unit 3 to a central storage unit 15, which preferably can be carried along by the patient 5. The data are transmitted to the central storage device 15 by using a wireless communication line 16. The central storage unit 15 can be a dedicated device, but also a mobile phone or another appliance configured accordingly.

The data unit 3 further comprises processing means 17, adapted for performing all tasks of processing and computing the received movement data as well as determining and assessing results and controlling internal or external notification means, which a described below in more detail. As processing means 17 preferably a microprocessor is used. The data unit 3 further comprises a computer program 18 adapted to be executed by the processing means 17 and further adapted for carrying out the steps of the inventive method, when the software is executed in the processing unit. The processing means 17 may comprise functional modules or units, which are implemented in the form of hardware, software or in the form of a combination of the two.

Preferably, the complete monitoring system 1, in particular all parts of the measuring unit 2 are carried by the monitored patient 5. The data unit 3 may be combined with one of the accelerometers 6, 6′, 9 or combined with the measuring device 2. Alternatively, measuring unit 2 and data unit 3 are located separately, e.g. the measuring unit 2 is worn by the patient 5 and the data unit 3 is carried along separately or located near the patient 5, e.g. implemented in a mobile phone or pocket computer or another handheld device.

All system means are adapted to carry out the method according to the present invention. All devices and units, in particular the measuring unit 2 and the data unit 3, are constructed and programmed in a way that the procedures for obtaining data, for data processing and system control run in accordance with the method of the invention.

The method according to the present invention is described with reference to FIG. 3. In a first step 100 movement data D₁, D₂ of the patient's 5 arm movements are obtained by the accelerometers 6, 6′, 9 of the measuring unit 2. The data unit 3 receives these movement data and the acquired data are analyzed by the data unit 3 in a second step 101 in order to distinguish cyclic movements (e.g. walking) and non-cyclic movements (e.g. eating). In other words the patient's 5 movements are classified by the data unit 3 according to cyclic and non-cyclic activities. The classification includes the determination of typical movement patterns, which occur during the standard movements, in particular different types of cyclic movements, e.g. walking, running etc. The term “cyclic” is used for activities such as walking, climbing stairs and running, that show movement patterns that periodically repeat themselves. Further information about the movement classification is given in: A. Schnitzer, O. Such, G. Schmitz, “Ein tragbares System für die Bewegungsanalyse zur Unterstützung des kardiologischen Dauermonitoring”, Proceedings DGBMT 2005: Biomedizinische Technik, Band 49, Ergänzungsband 2, p. 252, ISSN 0939-4990, which is incorporated herein by reference. Depending on the classification results a cyclicity threshold value C₀ is set by the data unit 3. Additionally, the patient 5 may state, by means of a user interface 19, which type of movement he is currently executing, e.g. as part of a therapy exercise. If applicable, non-cyclic activities such as lifting a cup or opening a door can be recognized by means of the data unit 3 using classification algorithms, as described in: H Junker, “Human Activity Recognition and Gesture Spotting with Body-Worn Sensors”, Hartung-Gorre 2005, pages 37-72, which is incorporated herein by reference.

The classification procedure uses measuring data, as depicted in FIGS. 4 and 5. FIG. 4 shows the raw accelerometer readout of two accelerometers 6, 6′ around the patient's 5 wrists during walking. Readouts from one channel are shown. In the upper part of the diagram movements 21 of the right arm and in the lower part of the diagram movements 22 of the left arm are illustrated. Note both the cyclicity of the motion and the symmetry. FIG. 5 shows the raw accelerometer readout of two accelerometers 6, 6′ around the patient's 5 wrists during the drinking of a cup of tea. As can clearly be seen the patient 5 moves his right arm, whereas his left arm rests. In this case no cyclicity and no symmetry can be observed.

Those measuring data are used by the data unit 3 in order to detect types of cyclic movements (like walking, running etc.). For this purpose, measuring data are compared to two-dimensional accelerometer histograms, wherein the vertical to horizontal change in acceleration is plotted over time. In other words, the difference between the sensor measurements is used to determine the deviation from an ideal movement, e.g. the deviation from an accelerometer histogram, which is used as a reference pattern. The accelerometer histograms are either known histograms, i.e. they are generated beforehand using a high number of similar patient movements and stored in the data unit 3, or self-generated, i.e. they are generated by the data unit 3 using prior measuring data of the present and/or other patients.

One example of such an accelerometer histogram 23 is shown in FIGS. 6 and 7, which illustrate the classification of two movements (in this case slow and fast stair climbing). The brightness of entries represents the magnitude of change of acceleration for a specific acceleration in x and y-direction. From those accelerometer histograms the types of patient movement are determined by the data unit 3.

At the same time the data unit 3 quantifies the movement's cyclicity C (step 102). This means, that the data unit 3 calculates a numerical value representing the movement's cyclicity C.

In a next step 103 the data unit verifies, whether the movement's cyclicity value C exceeds the cyclicity threshold value C₀. Depending on the implemented algorithm it might in another embodiment of the invention also be sufficient, that the movement's cyclicity value C equals the cyclicity threshold value C₀. In case the cyclicity threshold value C₀ is exceeded, i.e. there is a cyclic movement of the patient's limbs, the data unit 3 determines the movement's synchronicity S in the following step 104.

In case the cyclicity threshold value C₀ is not exceeded, the data unit 3 does not determine the movement's synchronicity S, since no cyclic movements are available.

If the data unit 3 determines the movement's synchronicity S, a synchronicity threshold value S₀ is set by the data unit 3. The synchronicity threshold value S₀ is set by the data unit 3 depending on the movement classification in step 101. For example, if the movement classification reveals that the patient 5 performs a certain movement, the synchronicity threshold value S₀ will be set to a level defined in a look-up table referring to this specific movement (and optionally referring to the specific patient 5). In other words, the classifications are taken into account when deciding on the synchronicity threshold S₀. The same holds true for the cyclicity threshold C₀. It will also depend on the type of movement.

In a further embodiment of the invention the synchronicity threshold value S₀ is—at least from a particular monitoring time on—dynamically adapted by means of the data unit 3 depending on prior and/or current synchronicity values S. Thus the therapeutic progresses of a specific patient 5 can be taken into account.

After the synchronicity threshold value S₀ is determined, it is compared by the data unit 3 to the measured actual synchronicity values S and evaluated, whether the desired synchronicity is reached or not (step 105).

For cyclic activities such as walking or running it is expected that the patient's 5 arms swing freely and with symmetrical amplitudes. This might not be the case when e.g. carrying a bag or holding the rail of a staircase. These types of movements will then be classified as non-cyclic movements and not be evaluated.

In a subsequent step 106 the data unit 3 stores the movement's synchronicity value S, if the desired synchronicity is reached. Alternatively, the data unit 3 stores the synchronicity value S to the data storage device 14 of the data unit 3 and notifies the patient 5 about the findings in step 107, if the actual movement of the patient 5 does not fulfil the expectations. In other words, the data unit 3 gives the patient 5 feedback on the degree of symmetry at which he executed his movements from the time on. Thus, the results can be immediately fed back to the patient 5. However, the results can also be stored, e.g. in the data storage device 14 or in the external central storage device 15 and analyzed offline by a therapist.

The notification will be carried out by means of audio and/or video signaling or mechanical/electrical stimulation of the corresponding limb. Notification means 25, in particular audio and video signaling means, are preferably included in the data unit 3 worn by the patient 5 or carried by the patient 5. Such signaling means may include e.g. loudspeakers, numbers, blinking lamps, e.g. LED lights, coloured indicators etc.

With the present invention a first, simple type of feedback can be given to the patient 5: The overall activity of a single limb can be evaluated (e.g. integration for 24 h) and a feedback to the patient 5 can be given, e.g. in the form of “You underused your left side considerably today, please take care of it.” or “Today you used your left side much more than yesterday. Try to go ahead just like this.” This would be a suitable approach for patients 5 that have a rather stable level of performance and should only be kept on track.

However, since a motion classification analysis is performed for every limb, a more elaborate feedback can be given: For cyclic types of movements, symmetry analyses are performed and feedback to the patient 5 can be more specific, e.g. “Please use your left arm more actively to support your walking movements”. In other words, if non-symmetrical use of the limbs is recognized, the monitoring system 1 reminds the patient 5 accordingly.

For non-cyclic activities such as taking a cup or opening a door it cannot be expected that the motion is symmetric. Therefore, these data have to be treated differently. Preferably the data unit 3 is adapted to evaluate, whether the patient 5 compensates for his weakness on one side by using only his strong side for activities like opening doors etc. If such a pattern is recognized, the data unit 3 reminds the patient 5 by means of the notification means 25.

The time of notification depends on the actual patient's movements. In other words, the patient 5 is not notified about progresses or regresses of a certain movement, e.g. about the activity status of his left arm during running, when he actually has just stopped running and now drinks a cup of tea. However, as soon the patient 5 starts running again, he might be notified accordingly.

Data analysis performed by the data unit 3 can be at different levels, depending on the therapeutic approach desired for the specific patient 5. The evaluation of the data can for example be done in comparison with historic data (trends, improvements), or in comparison with threshold levels that are defined by the therapist or that are set dynamically by the data unit 3 depending on prior or current measurement data and/or evaluation results.

The data can also be used to assess the results of a parallel therapy done conventionally. In particular the invention can also be used to support specific types of physical therapy, such as bilateral training and constraint-induced therapy. This may give feedback to the therapist on the outcome and quality of the therapist's work.

The invention can also be used to support and optimize training, especially in sports with long-term cyclic movements (i.e. running and swimming). Here the analysis of asymmetries can help optimizing the motion patterns (which is currently done on treadmills and with video analyses). The development of asymmetries during training may also be an early indicator for injuries (e.g. due to overuse/fatigue).

Some of the application paths require off-line analysis of the data (e.g. on a personal computer or notebook) and would involve professional support by a physical therapist and would thus require the respective infrastructure. In this case functions of the data unit are implemented by the external off-line analysis unit 26.

It will be evident to those skilled in the art that the invention is not limited to the details of the above illustrative embodiments, and that the present invention may be embodied in other specific forms without departing from the spirit or essential attributes thereof. The present embodiments are therefore to be considered in all respects to be illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 monitoring system
2 measuring unit
3 data unit
4 battery
5 patient
6 accelerometer
7 affected limb
8 healthy limb
9 accelerometer
10 (free)
11 data communication equipment
12 data communication equipment
13 data cable
14 data storage device
15 external central storage device
16 wireless communication line
17 processing means
18 computer program
19 user interface
20 (free)
21 right arm movement
22 left arm movement
23 accelerometer histogram
24 accelerometer histogram
25 notification means
26 external analysis unit
   100-106 method steps

1. A method of monitoring the functional use of limbs, comprising the steps of:
performing a permanent and unsupervised measuring of movement data, using a measuring unit (2), with at least parts of said measuring unit (2) being carried by the monitored patient (5),
analyzing said movement data,
determining the movement's cyclicity C,
determining the movement's synchronicity S depending on the movement's cyclicity C, and
storing the movement's synchronicity value S and/or notifying the patient (5) about the findings.

2. The method as claimed in claim 1, characterized in that the step of analyzing the movement data include classification of the patient's (5) movement.

3. The method as claimed in claim 2, characterized in that a cyclicity threshold value C₀ is set depending on the movement classification.

4. The method as claimed in claim 3, characterized in that, the movement's synchronicity S is determined only if the movement's cyclicity value C exceeds the cyclicity threshold value C₀,

5. The method as claimed in claim 2, characterized in that a synchronicity threshold value S₀ is set depending on the movement classification.

6. The method as claimed in claim 5, characterized in that the synchronicity threshold value S₀ is dynamically adapted depending on prior and/or current synchronicity values S.

7. The method as claimed in claim 1, characterized in that the movement's synchronicity S is compared with a synchronicity threshold value S₀ and the patient (5) is notified in case the movement's synchronicity value S falls below the synchronicity threshold value S₀.

8. The method as claimed in claim 1, characterized in that the kind of notification depends on the determined synchronicity value S.

9. The method as claimed in claim 1, characterized in that the time of notification depends on the actual patient's (5) movements.

10. A system (1) for monitoring the functional use of limbs, comprising a measuring unit (2), with at least parts of said measuring unit (2) being carried by the monitored patient (5), said measuring unit (2) being adapted to perform a permanent and unsupervised measuring of movement data, and a data unit (3), said data unit (3) being adapted
to analyze said movement data,
to determine the movement's cyclicity C,
to determine the movement's synchronicity S depending on the movement's cyclicity C, and
to store the movement's synchronicity value S and/or to notify the patient (5) about the result of said determination.

11. The system (1) as claimed in claim 10, characterized in that the measuring unit (2) comprises a number of inertial sensors, preferably accelerometer (6, 6′, 9) and/or magnetometer.

12. A computer program (18) for monitoring the functional use of limbs, using movement data obtained during a permanent and unsupervised measuring performed by a measuring unit (2), with at least parts of said measuring unit (2) being carried by the monitored patient (5), the program (18) comprising:
computer instructions to analyze said movement data,
computer instructions to determine the movement's cyclicity C,
computer instructions to determine the movement's synchronicity S depending on the movement's cyclicity C, and
computer instructions to store the movement's synchronicity value S and/or to notify the patient (5) about the findings,
when the computer program (18) is executed in a computer (17).
